(19) United States
(12) Patent Application Publication
(10) Pub. No.: US 2019/0115094 A1
(43) Pub. Date: Apr. 18, 2019

(54) MEDICAL DATA MANAGEMENT SYSTEM

(71) Applicant: American TrueCare, Inc., San Diego, CA (US)

(72) Inventors: Drew Markell, San Diego, CA (US); Oscar V. Medina, San Diego, CA (US); Jeremie Meyers, San Diego, CA (US)

(21) Appl. No.: 16/160,526

(22) Filed: Oct. 15, 2018

Related U.S. Application Data
(60) Provisional application No. 62/573,077, filed on Oct. 16, 2017.

(51) Int. Cl. G16H 10/60; G16H 50/20

(52) U.S. Cl. CPC G16H 10/60; G16H 50/20

(57) ABSTRACT

A medical data management system includes a medical data gathering device configured to obtain medical data from a patient and a data reporting device communicatively coupled to the medical data gathering device. A data center is communicatively coupled to the data reporting device over a data network, wherein the data center obtains data from the data reporting device, analyzes the data and transmits at least one notice related to the data.

### CROSS REFERENCE TO RELATED APPLICATION

This application claims priority to U.S. Application No. 62/573,077, filed on Oct. 16, 2017, the contents of which is fully incorporated by reference in its entirety.

### BACKGROUND

According to the recent statistics, roughly half of all adults suffer from at least one chronic disease, with a quarter of all adults suffering from two or more. In 2014, six chronic conditions were responsible for nearly 65 percent of all deaths. As a result, many adult patients are on some kind of medical treatment plan. Unfortunately, many such patients often do not comply with the treatment plan, which can result in expensive and unplanned visits to a hospital by the patients.

This can be an administrative and financial burden on health care providers as well as on the health care infrastructure that monitors and supports the healthcare providers and patients. Moreover, monitoring of such patients results in vast amounts of data, which can also be burdensome.

In view of the foregoing, there is a need for improved systems and methods of monitoring and encouraging patient compliance of medical treatment plans and also for managing the vast data associated with such treatment plans.

### SUMMARY

Disclosed is a healthcare data and communications system and method that is configured to communicatively link one or more patients with healthcare providers so as to enable improved outcomes and reduced costs. The type of healthcare provider can vary widely and can include, for example, physicians, insurance providers, accountable care organizations (ACOs), etc.

In an embodiment, the system is particularly suited for patients with high-cost, chronic conditions, although the type of patient can vary.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a schematic representation of a healthcare data system.

FIG. 2 shows a flow chart of an exemplary operation of the system.

FIG. 3 shows an exemplary user interface of the system.

FIG. 4 shows an exemplary user interface of a mobile device application for the system.

FIG. 5 shows a flow chart of an exemplary operation of the system.

FIG. 6 shows another example of a user interface.

### DETAILED DESCRIPTION

Disclosed is a healthcare data and communications system and method that is configured to communicatively link patients and care providers (such as physicians) together for improved outcomes and reduced costs. As described in detail below, the system is adapted to monitor specific daily activity of a patient via a device that is worn by the patient. The device is configured to remotely monitor a patient's activity and provide data that can assist in behavior modification of the patient. In this regard, the system links patient biometric devices to a remotely accessible data depository that can be used for data analytics and stratification. The result is reduced cost through reduced unnecessary utilization by patients with chronic conditions.

The disclosed system can be used by both patients and third party healthcare providers, such as, for example, provider groups (physicians), payers (insurance company's), and ACO's (Accountable Care Organizations). The system enables the third party providers to work with their patients for increasing compliance with a healthcare treatment plan by the patient. As mentioned, in an example embodiment, the system is particularly configured for use with patients that have been diagnosed with chronic conditions. The system enables an “extension” of the healthcare provider's office to increase the likelihood of continuous communication between the healthcare provider and the patient.

As described below, the system is configured to monitor specific patient data that can be analyzed pursuant to benchmarks. The physician (or other healthcare provider) can access a user interface that permits the physician to set such benchmarks for a patient. The system can analyze the data and provides actionable information back to the healthcare provider for next steps. This critical information keeps the physician informed and enables the provider to be proactive from a care standpoint instead of after an event that may lead to unnecessary utilization resulting in higher cost.

The system enables and provides a simplified connection of multiple disparate medical devices. A component of the system includes an application programming interface (API) that passes a measured data value (or other information depending on the device) through a gateway application to a compliant and secure platform to analyze and stratify specific biometric information. That information can then be stored and/or communicated to an appropriate clinician or service provider for intervention process.

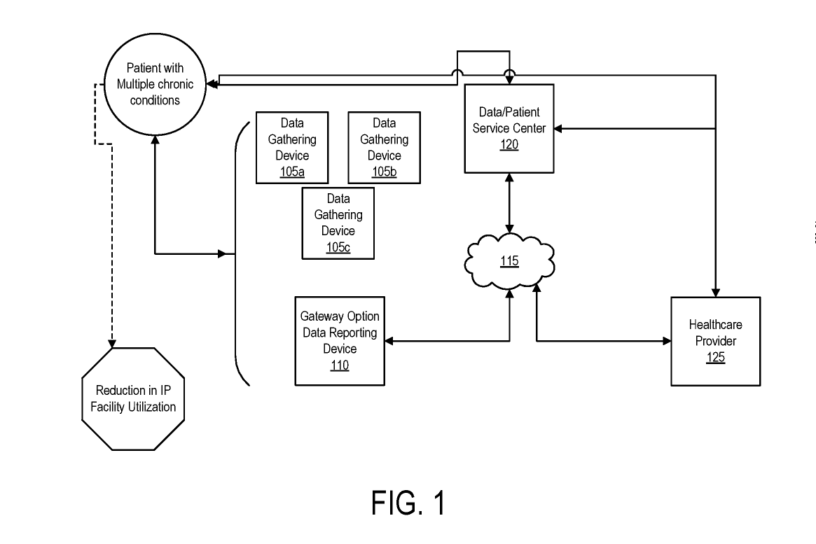

FIG. 1 shows a schematic representation of the system, which includes at least one data gathering device 105 (such as three data gathering devices 105a, 105b, and 105c) that are communicatively linked to a data reporting device 110 that is a device that can be worn by a patient, as described in more detail below. The patient is an individual that suffers from one or more chronic medical conditions. The data reporting device 110 is communicatively linked to a data network 115 such as the Internet. A data/patient service center 120 is also communicatively linked to the data network 115 for collection, sorting, reporting, and/or processing of data from the data gathering device 105 and data reporting device 115. A healthcare provider 125 is also communicatively linked to the network 115. The healthcare provider 125 can access data from the data center and can also receive one or more notifications, reminders, etc. from the data center related to management of patient care, as described below. The healthcare provider 125 can also remotely review analytics based on the data. The patient and the data/patient service center 120 are also communicatively linked, as are the patient and the healthcare provider 120.

With reference still to FIG. 1, the data gathering device 105 can be any type of device, such as a medical device, that is configured to obtain and gather data related to a patient. The data gathering device 105 can be any of a variety of devices for recording and obtaining data related to a patient's health, such as, for example, a weight scale, a blood pressure cuff, a glucometer, an activity sensor, etc. The data gathering device 105 can communicate and can be coupled to via a wired or wireless connection to the data reporting device 110. The system can include any quantity of data gathering devices 105 and data reporting devices 110 that are linked to the patient or multiple patients.

The data reporting device 110 can be, for example, a wearable device such as a smart phone or smart watch. The data reporting device 110 is configured to automatically interact with and obtain data from the data gathering device 105 such as upon the data reporting device 110 coming into a predetermined proximity of the data gathering device 105. In an embodiment, a user can also manually cause the data reporting device 110 to obtain data from the data gathering device 105.

As mentioned, the data reporting device 110 is configured to communicate such gathered data via the network 115 to the data center 120, where the data can be stored and analyzed. The healthcare provider 125 can access the data via the network 115 and can also analyze the data, such as via a local user interface. In addition, the system is configured such that the data center (or other entity) can provide updates, reminders, and analytics regarding the patient to the healthcare provider.

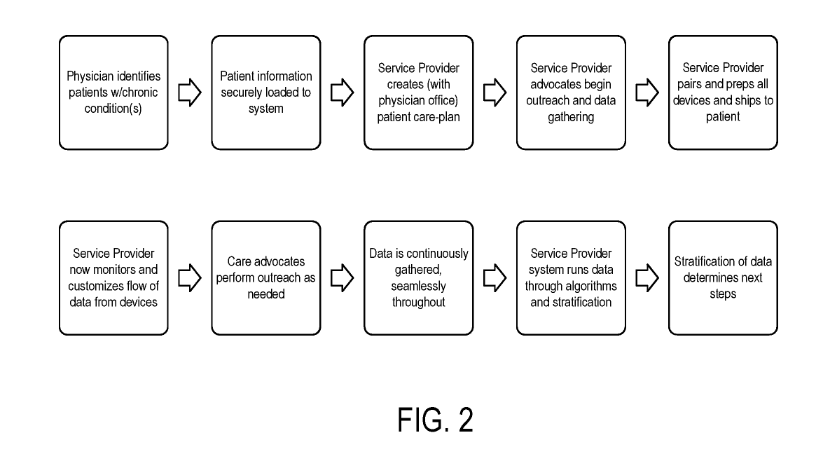

FIG. 2 shows a flow chart of an exemplary process for operation of the system. In a first step, a healthcare provider, such as a physician, identifies one or more patients with at least one chronic condition. Data regarding the patient, such as identifying data and data related to the condition, is then loaded to the system such as to the data center 120. A service provider with assistance from the physician then creates a care-plan for medical care of the patient.

Next, the service provider begins outreach and data gathering wherein the service provider gathers data from the patient. In a next step, the service provider pairs and preps relevant devices, such as the data gathering device(s) 105 and the data reporting device 110 for the patient. In this regard, the patient can then put the devices to use such as by using the data gathering devices to gather relevant data such as weight, blood glucose level, blood pressure, etc. As mentioned, the data gathering devices communicate such data to the data reporting device, which transmits the data to the data center. The service provider now monitors and customizes flow of data from the devices to the data center. The service provider can configure the data center to automatically analyze, tabulate, and monitor the data so that the physician can perform outreach as needed to the patient based on the analysis of the gathered data over time. The data is continuously gathered, seamlessly throughout a period of time and the service provider can run the data through algorithms and stratification for presentation and use by the physician. The physician can then use the stratification to determine any next steps for the patient, such as to recommend a change in a medical care plan for the patient.

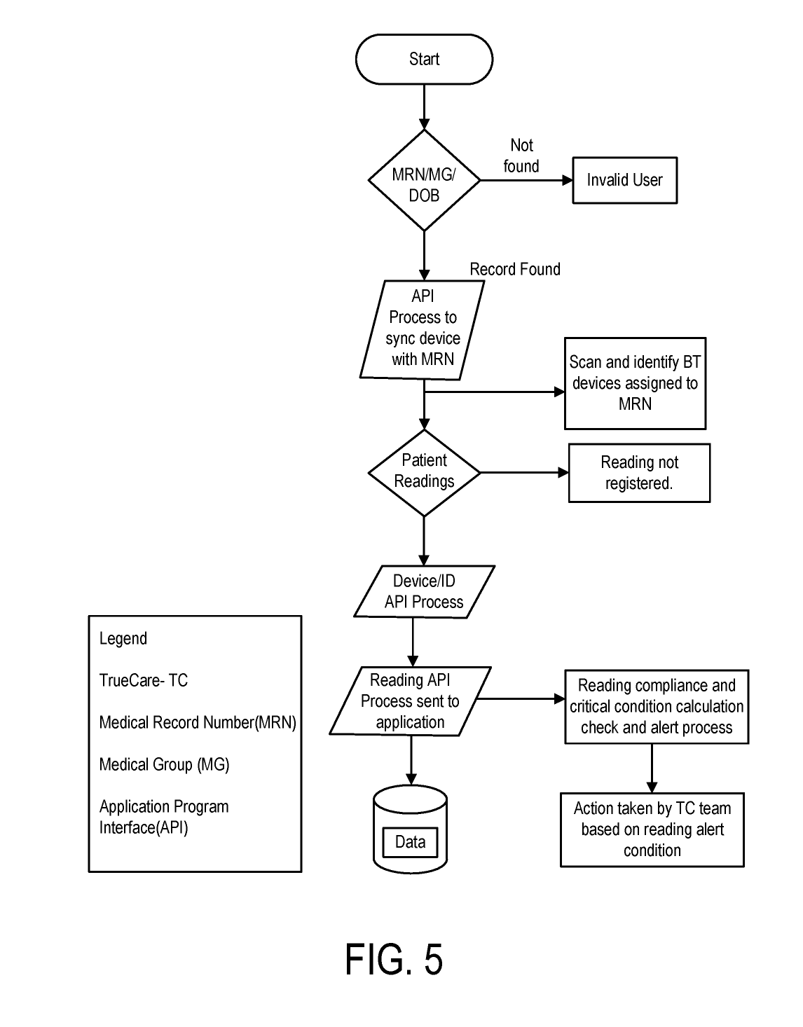

FIG. 5 shows a flow chart of another exemplary process for operation of the system. In a first step, the system obtains information regarding a user, such as a patient. The information can vary and can include, for example, a Medical Record Number (MRN), a Medical Group (MG), and a date of birth (DOB). If the information is not found in the system database, then the user is identified as invalid. In a next step where the information is found, the system implements an API to sync the data gathering device or data reporting device with the MRN. In connection with this step, the API may scan and identify any devices assigned to the MRN. The system can then obtain patient readings such as data from the data gathering device(s).

Next, the system will collect any device identifiers such as for the data gathering device and/or data reporting device. The API then sends any gathered data to a local application on the data gathering device and stores such data. The system can also implement analyses of the data for reporting of critical condition(s) to the patient and/or the healthcare provider. A service provider (TC) can also take action such as to send a notification or alert to the user.

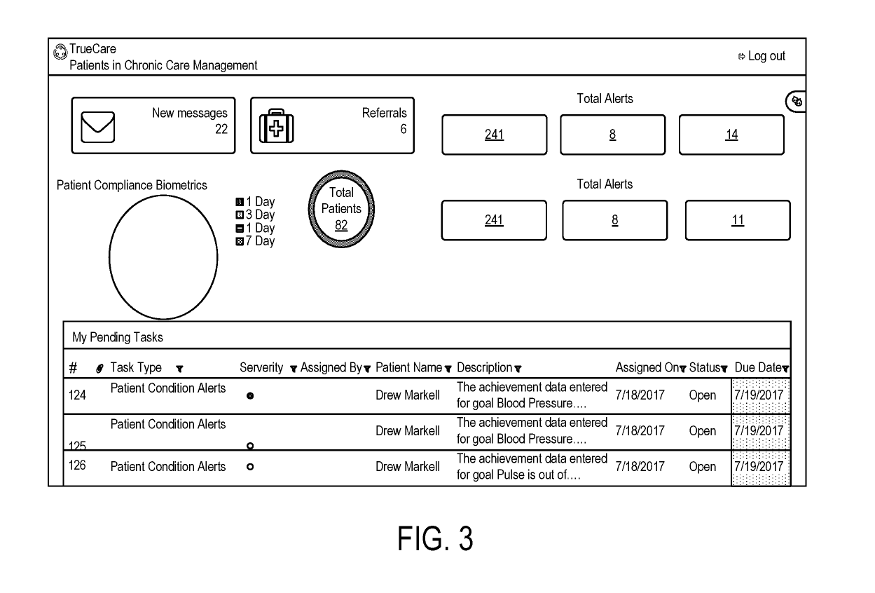

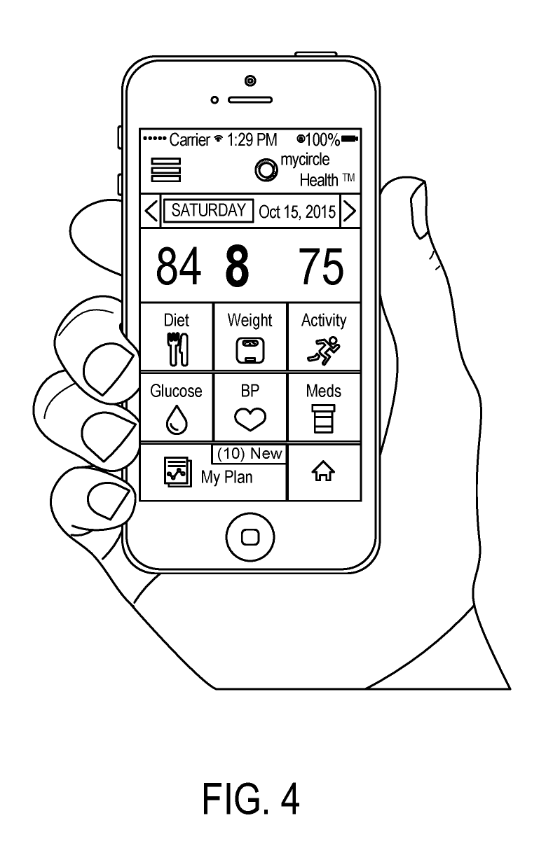

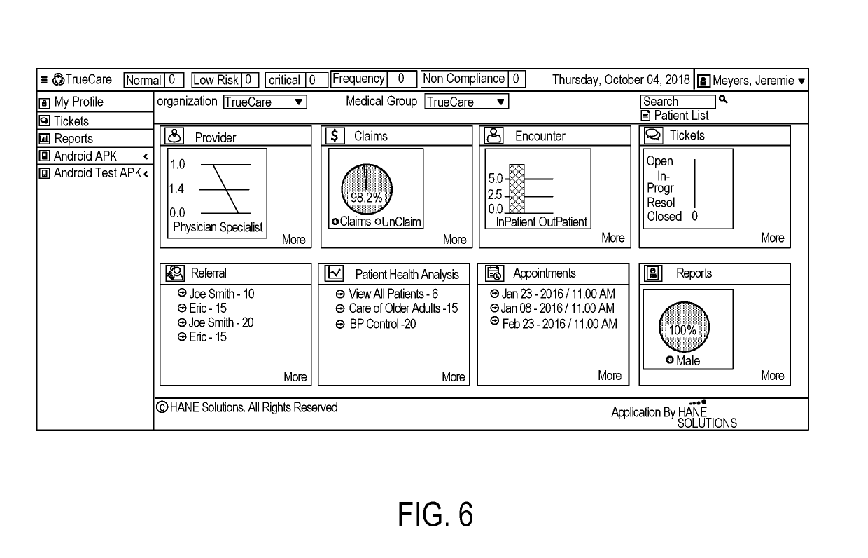

FIG. 3 shows an example of a user interface that a service provider may access remotely in order to analyze or otherwise use the data related to the patient. FIG. 4 shows an example user interface of a mobile device application, such as a phone application, by which the patient can monitor his or her data. FIG. 6 shows another example of a user interface.

One or more aspects or features of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system. The implementations can also include at least one input device (e.g., mouse, touch screen, etc.) and at least one output device.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a computer processor and a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

As will be apparent to those of skill in the art upon reading this disclosure, each of the individual embodiments described and illustrated herein has discrete components and features which may be readily separated from or combined with the features of any of the other several embodiments without departing from the scope of the subject matter described herein. Any recited method can be carried out in the order of events recited or in any other order which is logically possible.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

1. A medical data management system, comprising:
a medical data gathering device configured to obtain medical data from a patient;
a data reporting device communicatively coupled to the medical data gathering device;
a data center communicatively coupled to the data reporting device over a data network, wherein the data center obtains data from the data reporting device, analyzes the data and transmits at least one notice related to the data.

2. A system as in claim 1, wherein the notice is transmitted to a healthcare provider.

3. A system as in claim 1, wherein the notice is transmitted to a patient.

4. A system as in claim 1, wherein the notice is a reminder to perform an action related to the data.

5. A system as in claim 1, wherein the data reporting device is configured to automatically interact with and obtain data from the data gathering device.

6. A system as in claim 1, wherein the data reporting device is configured to automatically interact with and obtain data from the data gathering device upon the data reporting device coming into a predetermined proximity of the data gathering device.

7. A system as in claim 1, wherein the medical data gathering device is coupled to the data reporting device via a wired connection.

8. A system as in claim 1, wherein the medical data gathering device is coupled to the data reporting device via a wireless connection.

9. A system as in claim 1, wherein the medical data gathering device is a medical device.

10. A system as in claim 1, wherein the medical data gathering device is a weight scale, a blood pressure cuff, a glucometer, or an activity sensor.

11. A system as in claim 1, wherein the data reporting device is a wearable device.

12. A system as in claim 1, wherein the data reporting device is a smart phone, a smart watch.

13. A system as in claim 1, wherein the data relates to a chronic medical condition.
